(19) United States
(12) Patent Application Publication
(10) Pub. No.: US 2026/0053538 A1
(43) Pub. Date: Feb. 26, 2026

(54) DYNAMIC SYNDESMOSIS IMPLANT SYSTEMS AND METHODS

(71) Applicant: Paragon 28, Inc., Englewood, CO (US)

(72) Inventors: Kyle James HARTSON, Denver, CO (US); Kevin FARLEY, Denver, CO (US)

(73) Assignee: Paragon 28, Inc., Englewood, CO (US)

(21) Appl. No.: 19/287,311

(22) Filed: Jul. 31, 2025

Related U.S. Application Data
(63) Continuation of application No. PCT/US2024/014442, filed on Feb. 5, 2024.
(60) Provisional application No. 63/483,261, filed on Feb. 3, 2023.

(51) Int. Cl. A61B 17/86; A61B 17/04; A61B 17/68; A61B 17/80

(52) U.S. Cl. CPC A61B 17/863; A61B 17/8061; A61B 17/864; A61B 2017/0409; A61B 2017/0414; A61B 2017/044; A61B 2017/681

(57) ABSTRACT

An implant system for stabilization of bones and/or tissue. The system includes a dynamic bone anchor and at least one tether and at least one secondary tether anchor. The anchor includes a cannulated main body having a proximal head portion, a distal end portion and a body portion. The anchor also includes a piston member and a resilient bumper positioned within the cannulation of the main body. The body portion also includes bone engagement projections extending about outer sides of the body portion and an outer groove extending through the bone engagement projections. The piston member has a first lateral through hole, and the distal end portion has a second lateral through hole that is in communication with the first lateral through hole. The body portion further includes a lateral aperture that is in communication with the cannulation and is substantially aligned with the outer groove.

### CROSS REFERENCE TO RELATED APPLICATION

This application is a continuation of International Patent Application No. PCT/US2024/014442, filed on Feb. 5, 2024, and entitled “Dynamic Syndesmosis Implant Systems and Methods,” which claims benefit of priority of U.S. Provisional Patent Application No. 63/483,261 , filed on Feb. 3, 2023, and entitled “Dynamic Syndesmosis Implant Systems and Methods” the disclosure of which is hereby incorporated herein by reference in its entirety.

### TECHNICAL FIELD

The present disclosure relates generally to orthopedic surgery related to dynamic stabilization of bones and/or tissue. More specifically, but not exclusively, the present disclosure relates to implant devices, systems, and methods that achieve dynamic stabilization of a syndesmosis joint.

### BACKGROUND OF THE INVENTION

A syndesmosis joint is a fibrous joint where two bones are connected by strong ligaments or membrane. The distal tibiofibular syndesmosis, between the fibula and tibia, is formed by three major ligaments: the anterior inferior tibiofibular ligament (AITFL), the posterior inferior tibiofibular ligament (PITFL), and the interosseous tibiofibular ligament (ITFL). A fourth ligament, the inferior transverse tibiofibular ligament, is congruent with the PITFL, but sometimes considered a separate ligament. While technically the syndesmosis is the joint, most literature describes a syndesmosis injury as affecting the syndesmotic ligaments,

Syndesmotic injuries most typically occur to the distal tibiofibular syndesmosis, and result from trauma (such as, but not limited to, sports injuries). Syndesmotic injuries can occur as a purely ligamentous injury, or in combination with a bone fracture, where the syndesmotic ligaments become disrupted, separated, or injured.

The current standard of care for syndesmotic injuries involves either rigid fixation with a screw, or a suture/tether-based constraint (stabilization) across the entire width of the syndesmosis joint. Rigid screw-based fixation is simple to implant and stabilizes the joint, but fails to allow any motion at all, as would normally exist physiologically. This limits the patient's range of motion, and unpredictable screw failure locations can result in damage to existing bone and/or patient pain.

Currently commercially available suture/tethered-based syndesmosis stabilization/constraint implant systems allow for limited motion of the joint, but fail to mimic the intact ligament structures of the syndesmosis in terms of attachment location and distance between the bones of the joint (e.g., the tibia and fibula). These current stabilization/suture-based syndesmotic implants typically involve drilling through the joint bones in their entirety (e.g., drilling through the fibula and the tibia lateral to medial for distal tibiofibular syndesmosis), with a first component of the implant being positioned on a surface of a first bone (e.g., the lateral surface of the fibula), a second component positioned on a surface of a second bone (e.g., the medial surface of the tibia), and the suture or other elastic/resilient-type tether extending therebetween.

Different injuries to the syndesmotic and surrounding soft tissue structures may be best addressed with different treatment options. For example, some injuries are best addressed with constant, rigid fixation through the syndesmotic space, whereas other injuries may be best treated with a progression from rigid fixation to dynamic fixation. Further, some injuries are best treated without rigid fixation at all, and are instead best addressed with completely non-rigid, suture-based systems.

Thus, new and improved dynamic syndesmosis stabilization devices, systems, and methods are needed to overcome the above-noted drawbacks of the currently available solutions for addressing syndesmotic injuries.

### SUMMARY OF THE INVENTION

The present disclosure is directed toward syndesmotic implants, systems and methods for use in dynamic stabilization of a syndesmotic joint (i.e., constrained and controlled movement and/or forces in the joint). While the implants, systems and methods disclosed herein (and described in detail below) are particularly advantageous for use in, and dynamic stabilization of, distal tibiofibular syndesmosis, the implants and methods may be configured and utilized for use with any joint, and in particular any syndesmotic joint. The dynamic stabilization syndesmotic implants, systems and methods provide an advantageous relatively short suture/tether length which does not extend entirely through the syndesmotic joint bones. For example, with respect to dynamic stabilization of the distal tibiofibular syndesmosis, the dynamic stabilization syndesmotic implants, systems and methods are advantageously configured such that the implant does not extend to the medial portion of the tibia. The suture/tether of the dynamic stabilization syndesmotic implants, systems and methods thus advantageously better resists stretching from a strength/load perspective and time perspective.

The dynamic stabilization syndesmotic implants, systems and methods also minimize the amount of bone removal (e.g., bone drilling) of the syndesmotic joint bones for through holes to accommodate the suture/tether of the system. For example, with respect to dynamic stabilization of the distal tibiofibular syndesmosis, the dynamic stabilization syndesmotic implants, systems and methods advantageously limits the amount of bone removal from the tibia. The suture/tether of the dynamic stabilization syndesmotic implants, systems and methods thus advantageously minimize the number of bone openings and the total amount of bone removal/disturbance, which may not be completely filled/plugged by the suture/tether of the implants, systems and methods.

The dynamic stabilization syndesmotic implants, systems and methods also provide for numerous configuration and implantation that are able to treat numerous different injuries to the syndesmotic and surrounding soft tissue structures. The dynamic stabilization syndesmotic implants, systems and methods provide intraoperative flexibility with options including rigid fixation, progressive rigid fixation, dynamic stabilization, and suture-based stabilization. For example, the dynamic stabilization syndesmotic implants, systems and methods provide two different bone anchor types: static bone anchors that simply act as a rigid steadfast anchor point for a suture or tether, and dynamic bone anchors that constrain and/or control movement of the suture (and thereby movement of the respective bones/tissue).

In one aspect, the present disclosure provides an implant system for dynamic stabilization of a bones and/or tissue, comprising: a dynamic bone anchor; and at least one tether extending through and between the dynamic bone anchor and at least one secondary tether anchor. The dynamic bone anchor comprises: a cannulated main body comprising: a proximal head portion, a distal end portion and a body portion extending between the proximal head portion and the distal end portion; a piston member positioned within the cannulation of the main body; and a resilient bumper positioned within cannulation of the body portion between at least a portion of the piston member and a retaining feature within the cannulation of the body portion. The body portion comprises bone engagement projections extending about outer sides thereof. The body portion comprises an outer groove extending through the bone engagement projections. The piston member comprises a first lateral through hole, and the distal end portion comprises a second lateral through hole that is in communication with the first lateral through hole. The body portion comprises a lateral aperture that is in communication with the cannulation thereof and is substantially aligned with the outer groove.

In some embodiments, lateral aperture is positioned within the outer groove. In some embodiments, first lateral through hole is substantially aligned with the outer groove. In some embodiments, proximal head portion comprises at least one bone engaging projection extending about outer sides thereof. In some embodiments, the distal end portion is void of a bone engaging projection.

In some embodiments, piston member is movably retained within the cannulation of the main body. In some such embodiments, a distal portion of the piston member comprises an elongated slot, and wherein a pin member extends from a wall portion of the main body into the elongated slot to limit the travel of the piston member through the cannulation. In some such embodiments, the distal portions of the piston member extend into cannulation of the bumper.

In some embodiments, the retaining feature comprises an interior wall of the body portion. In some such embodiments, the resilient bumper positioned within cannulation of the body portion between a distal portion of the bumper and the interior wall.

In some embodiments, the cannulation of the main body extends to top side of the head portion such that the cannulation forms a top side opening at the top side of the head portion that is in communication with the cannulation and the lateral aperture. In some embodiments, a portion of the tether extends through the top side opening, through the cannulation of the main body to the lateral aperture, through the lateral aperture, and along the outer groove to the second lateral through hole. In some embodiments, a portion of the tether extends along the main body over the bone engagement projections, laterally through a first side of the second lateral through hole, laterally through the first lateral through hole, and laterally through a second side of the second lateral through hole.

In some embodiments, the system comprises a plurality of the dynamic bone anchors. In some embodiments, the at least one secondary tether anchor comprises a plurality of secondary tether anchors.

In some embodiments, the at least one secondary tether anchor comprises at least one soft bone anchor that is configured to deform into an enlarged shape upon tensioning of the tether extending thereto.

In some embodiments, the at least one secondary tether anchor comprises at least one bone plate. In some such embodiments, the at least one secondary tether anchor comprises at least one anchor cap configured to couple with an aperture of the at least one bone plate, the at least one anchor cap comprising an open cavity with at least one tether retention cross-member within the open cavity. In some other such embodiments, the at least one secondary tether anchor comprises at least one static bone anchor configured to couple with an aperture of the at least one bone plate, the at least one static bone anchor comprising an open cavity with at least one tether retention cross-member within the open cavity.

In some embodiments, the at least one secondary tether anchor comprises at least one secondary static bone anchor configured to couple directly within a bone and not with a bone plate, the at least one secondary static bone anchor comprising an open cavity with at least one tether retention cross-member within the open cavity. In some embodiments, the tether comprises a plurality of tethers, and wherein at least one the plurality of teachers is configured as a tether passer comprising at least one loop or through hole. In some embodiments, the system further comprises an instrument coupled with a dynamic bone anchor of the at least one dynamic bone anchor configured to insert the dynamic bone anchor into a bone. In some such embodiments, the tether is coupled to a tensioning handle of the instrument that is removably and rotatably coupled to a housing body, and wherein the instrument is configured such that rotation of the tensioning handle wraps the tether about a portion of the tensioning handle and, ultimately, tensions the tether. In some such embodiments, the tether extends through the first and lateral through holes, and wherein the tether extends from the cannulation of the head portion between the dynamic bone anchor and an insertion portion of the instrument that houses the dynamic bone anchor and out a distal end of the insertion portion. In some such embodiments, the portion of the tether that extends between the dynamic bone anchor and the insertion portion to the distal end of the insertion portion substantially equals a longitudinal length of a portion of the dynamic bone anchor that is housed in the insertion portion from the distal end of the insertion portion.

In another aspect, the present disclosure provides a method of dynamically stabilizing a distal tibiofibular syndesmosis joint. The method comprises obtaining any of the implant systems disclosed herein; implanting the dynamic bone anchor into one of a fibula or a tibia of the joint; and implanting the at least one secondary tether anchor into the other of the fibula or the tibia. Implanting the dynamic bone anchor comprises tensioning the tether between the dynamic bone anchor and the at least one secondary tether anchor to dynamically stabilize the joint.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 illustrates a perspective view of an exemplary dynamic tether-tensioning bone anchor of a stabilization implant system, in accordance with an aspect of the present disclosure;

FIG. 2 illustrates another perspective view of the dynamic tether-tensioning bone anchor of FIG. 1, in accordance with an aspect of the present disclosure;

FIG. 3 illustrates another perspective view of the dynamic tether-tensioning bone anchor of FIG. 1, in accordance with an aspect of the present disclosure;

FIG. 4 illustrates another perspective view of the dynamic tether-tensioning bone anchor of FIG. 1, in accordance with an aspect of the present disclosure;

FIG. 5 illustrates a perspective exploded view of the dynamic tether-tensioning bone anchor of FIG. 1, in accordance with an aspect of the present disclosure;

FIG. 6 illustrates another perspective exploded view of the dynamic tether-tensioning bone anchor of FIG. 1, in accordance with an aspect of the present disclosure;

FIG. 7 illustrates a cross-sectional view the dynamic tether-tensioning bone anchor of FIG. 1, in accordance with an aspect of the present disclosure;

FIG. 8 illustrates another perspective view of the dynamic tether-tensioning bone anchor of FIG. 1 with a portion being transparent, in accordance with an aspect of the present disclosure;

FIG. 9 illustrates a side view of the dynamic tether-tensioning bone anchor of FIG. 1 implanted into a bone, in accordance with an aspect of the present disclosure;

FIG. 10 illustrates a side cross-sectional view of the dynamic tether-tensioning bone anchor of FIG. 1 implanted into a bone, in accordance with an aspect of the present disclosure;

FIG. 11 is a top, front perspective view of an instrument for implanting and tensioning the dynamic tether-tensioning bone anchor of FIG. 1, in accordance with an aspect of the present disclosure;

FIG. 12 is a rear, top perspective view of the instrument of FIG. 11 used for implanting and tensioning the dynamic tether-tensioning bone anchor of FIG. 1, in accordance with an aspect of the present disclosure;

FIG. 13 is an enlarged view of the front end of the instrument of FIG. 11 used for implanting and tensioning the dynamic tether-tensioning bone anchor of FIG. 1, in accordance with an aspect of the present disclosure;

FIG. 14 is a cross-sectional side perspective view of the instrument of FIG. 11 used for implanting and tensioning the dynamic tether-tensioning bone anchor of FIG. 1, in accordance with an aspect of the present disclosure;

FIG. 15 is a cross-sectional top perspective view of the instrument of FIG. 11 used for implanting and tensioning the dynamic tether-tensioning bone anchor of FIG. 1, in accordance with an aspect of the present disclosure;

FIG. 16 is a top view of the instrument of FIG. 11 used for implanting and tensioning the dynamic tether-tensioning bone anchor of FIG. 1, in accordance with an aspect of the present disclosure;

FIG. 17 is an enlarged, cross-sectional view of the front end of the instrument of FIG. 11 used for implanting and tensioning the dynamic tether-tensioning bone anchor of FIG. 1, in accordance with an aspect of the present disclosure;

FIGS. 18A-18D illustrate perspective views of a variety of stabilization implant systems utilizing the tether-tensioning bone anchor of FIG. 1, in accordance with an aspect of the present disclosure;

FIG. 19 illustrates a perspective view of a soft bone anchor that may be utilized with the tether-tensioning bone anchor of FIG. 1, in accordance with an aspect of the present disclosure;

FIG. 20 illustrates a perspective view of a static bone anchor that may be utilized with the tether-tensioning bone anchor of FIG. 1, in accordance with an aspect of the present disclosure;

FIG. 21 illustrates a perspective view of another static bone anchor that may be utilized with the tether-tensioning bone anchor of FIG. 1, in accordance with an aspect of the present disclosure;

FIGS. 22-23 illustrate perspective views of a plate interfacing anchor that may be utilized with the tether-tensioning bone anchor of FIG. 1, in accordance with an aspect of the present disclosure; and

FIGS. 24-25 illustrate side perspective views of another static bone anchor that may be utilized with the tether-tensioning bone anchor of FIG. 1, in accordance with an aspect of the present disclosure.

### DETAILED DESCRIPTION OF THE INVENTION

Generally stated, disclosed herein are devices and systems for achieving bone stabilization. Further, methods for using the devices and systems to achieve ligament fixation are discussed.

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior”means a direction below another object or structure.

Similarly, positions or directions may be used herein with reference to anatomical structures or surfaces. For example, as the current implants, devices, instrumentation and methods are described herein with reference to use with the bones of the ankle/leg, the bones of the foot, ankle and lower leg may be used to describe the surfaces, positions, directions or orientations of the implants, devices, instrumentation and methods. Further, the implants, devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the implants, devices, instrumentation and methods, and the aspects, components, features and the like thereof, described herein with respect to the right leg may be mirrored so that they likewise function with the left leg. Further, the implants, devices, instrumentation and methods, and the aspects, components, features and the like thereof, disclosed herein are described with respect to the leg for brevity purposes, but it should be understood that the implants, devices, instrumentation and methods may be used with other bones of the body having similar structures.

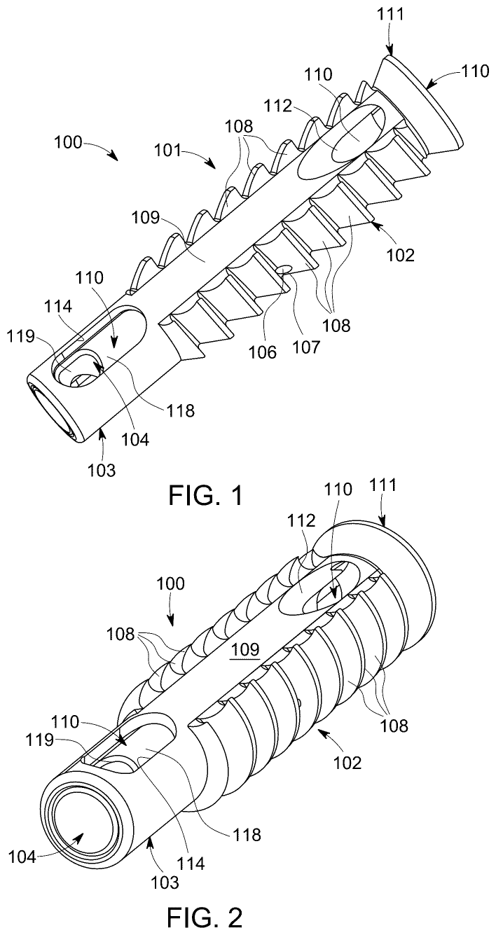

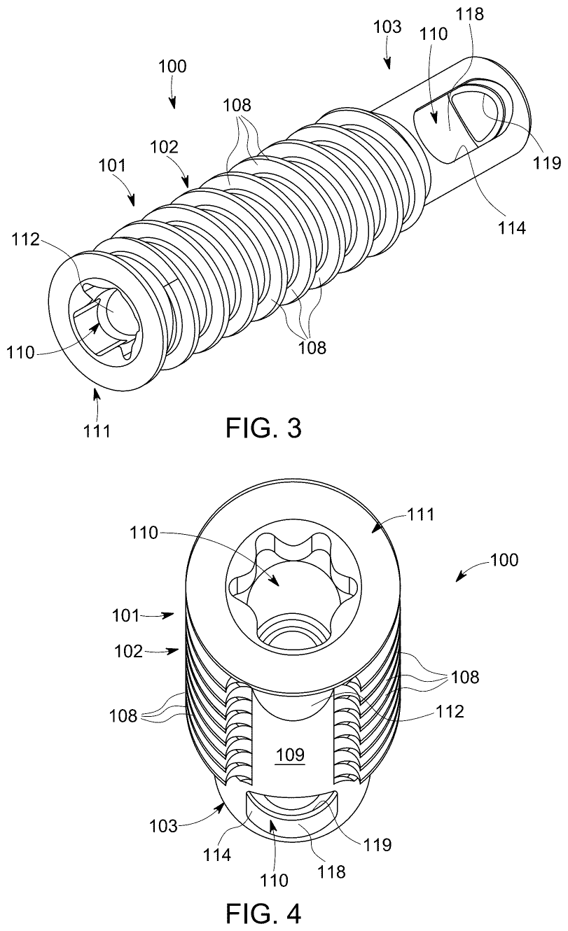

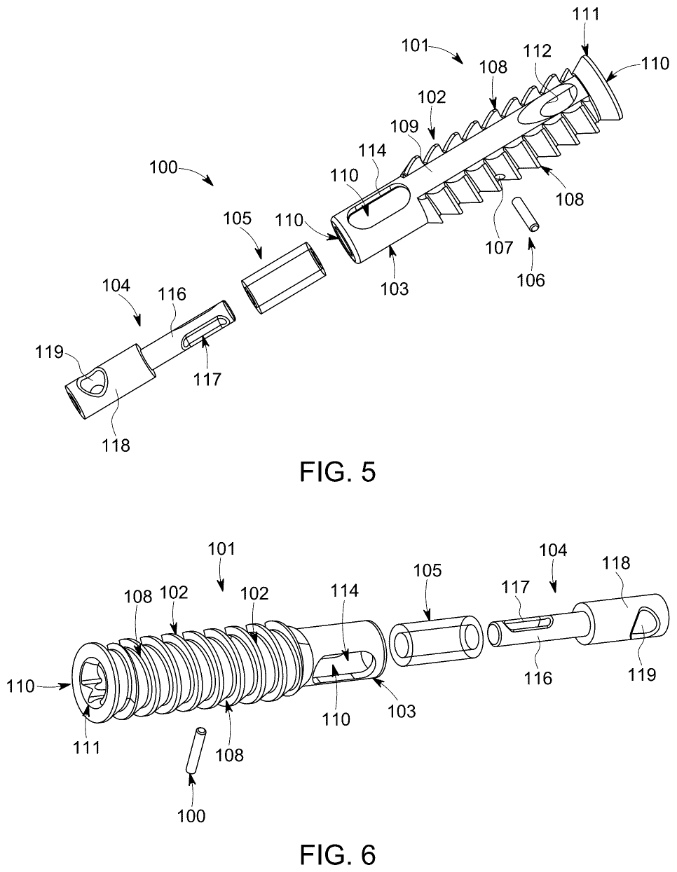

Referring to the drawings, wherein like reference numerals are used to indicate like or analogous components throughout the several views, and with particular reference to FIGS. 1-10 there is illustrated an implant system including at least one dynamic bone anchor. The implant system may be, for example, configured to heal syndesmotic ligaments post-operatively and/or stabilize syndesmotic joint, such as but not limited to a distal tibiofibular syndesmosis. The at least one dynamic bone anchor is configured to selectively constrain motion between syndesmotic bones (e.g., in all directions) to allow for one or more syndesmotic ligaments between the distal the fibula and tibia extending therebetween to heal and/or to stabilize the syndesmotic joint. The at least one dynamic bone anchor allows for only a limited amount of relative movement/motion between bones in which it is implanted or installed, and such relative movement/motion and/or forces acting between the bones is be controlled (e.g., resisted/inhibited and/or encouraged/enhanced).

The components and portions of the implant system may be made of, for example, titanium, stainless steel, polymers, polyester, polypropylene, UHMWPE, thermoplastic (e.g., thermoplastic urethane), bio-resorbable materials or any other biocompatible material so that the implant system is configured to be implanted into a mammalian patient, such as a human patient.

The implant system provides for allow dynamic stabilization and/or fixation of a joint between two or more bones (either naturally distinct bones or bone portions/segments), such as bones of a syndesmotic joint (e.g., a distal fibula and tibia of a distal tibiofibular syndesmosis). The implant system is configured to provide dynamic stabilization through constrained and/or controlled relative motion between the bones at least one dynamic bone anchor, and/or suture based stabilization or fixation with at least one secondary tether-bone anchors and/or bone plates. In some embodiments, the area of allowed constrained controlled motion provided by the implant system may be set in a space or gap between adjacent bones or bone segments (such as the syndesmotic joint/space between the distal the fibula and tibia).

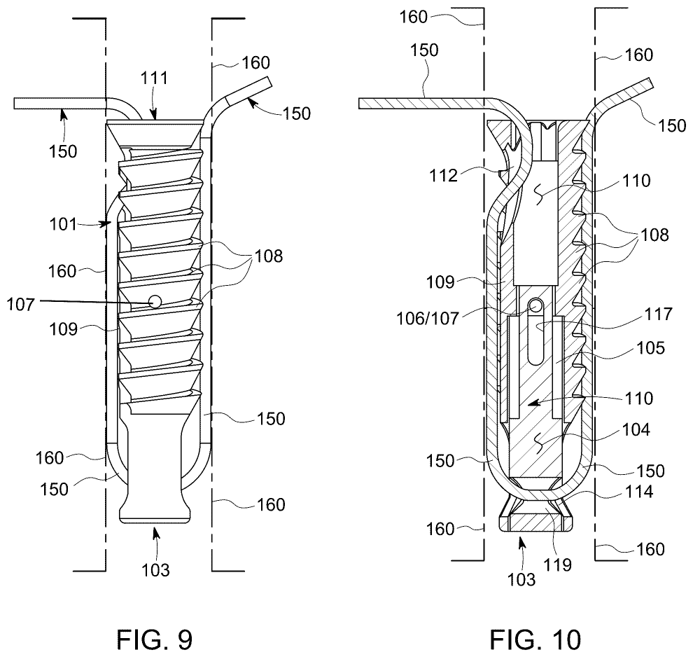

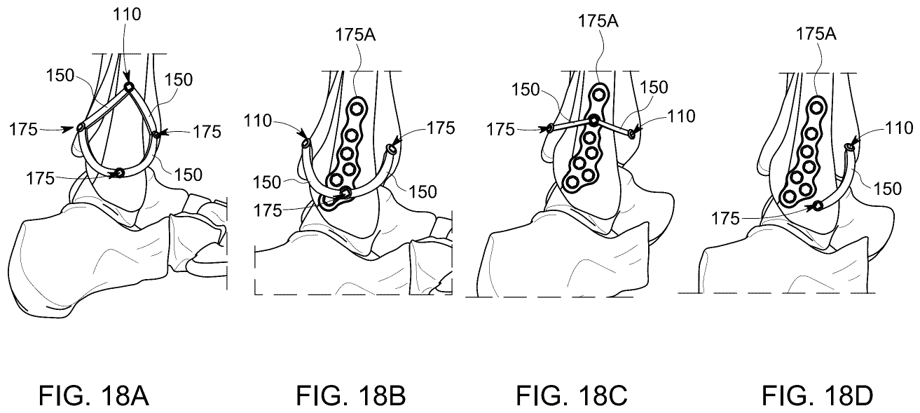

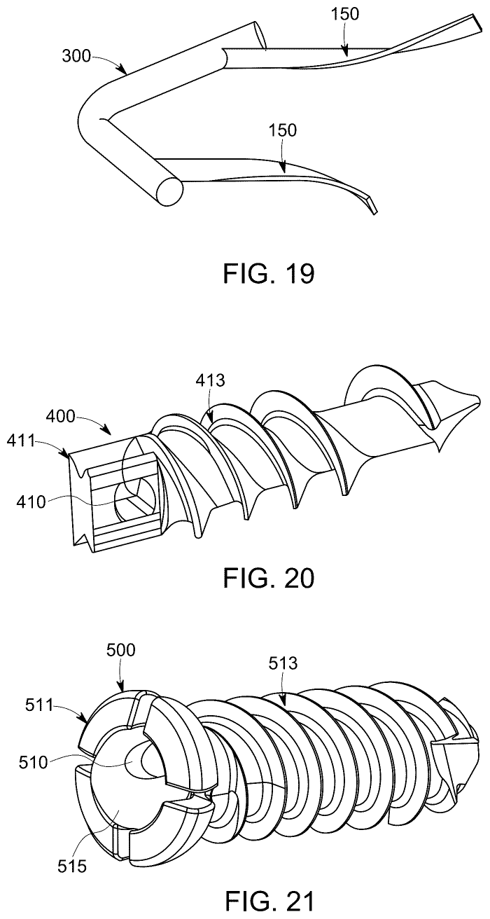

As shown in FIGS. 9, 10, 18 and 18A-189D, the implant system includes a flexible suture or tether 150 as a constraint and/or tension member that extends through portions of, and from, a dynamic bone anchor 100. As also shown in FIGS. 9 and 10, a portion of the tether 150 passes through, or is otherwise coupled with, the dynamic bone anchor 100. As shown in FIGS. 18A-189D, the tether 150 may extend from the dynamic bone anchor 100 to one or more secondary bone anchors 175, such as those shown in FIGS. 19-25 and discussed below.

The dynamic bone anchor 100 and the at least one secondary anchor 175 are configured to respectively be coupled to and implanted at least partially within first and second bones, such as bones of a syndesmotic joint (e.g., a distal fibula and tibia, respectively, of a distal tibiofibular syndesmosis), with the tether 150 extending between and coupled to the dynamic bone anchor 100 and the secondary anchor 175.

As also explained below, a portion of the tether 150 is retained in the dynamic bone anchor 100 via an adjacent portion of the tether 150 being compressed or pinched between an outer surface (which may include notches, ridges, or other textures configured to facilitate retention of the tether 150) of the dynamic bone anchor 100 and an adjacent bone/bone material/tissue, as shown in FIGS. 9 and 10. The dynamic bone anchor 100 includes a dynamic element that acts against the portion of the tether 150 that passes a distal portion 103 of the dynamic bone anchor 100, as explained further below. The dynamic bone anchor 100 of the implant system thereby provides dynamic stabilization of one or more joints.

The tether 150 may be configured to mimic the function, location and/or length of an interosseous ligament, for example. The tether 150 may be of any thin, long and substantially freely manually flexible configuration or structure, such as a suture, strand, cable or string-like configuration. The tether 150 may be, for example, a biomedical suture or tether (e.g., a stranded cerclage cable), or similar construct. In some embodiments, the tether 150 may be formed of, for example, polymer, polyester, polypropylene or UHMWPE suture, strands or filaments, braids thereof, or a like material, as known by one of ordinary skill in the art. In some aspects, at least a portion of the tether 150 may be bioresorbable. The tether 150 may be, for example, a suture (e.g., a braided suture), such as a single cross-section strand of suture or multiple loops of suture. For example, the tether 150 may be a UHMWPE and polypropylene co-braid suture.

The tether 150 may or may not be elastically axially/longitudinally stretchable or deformable. In some embodiments, the system may include a plurality of tethers. In some embodiments, at least one tether may be configured as a tether passer. In such embodiments the tether 150 may have one or more openings or gaps along its length that is/are configured to allow another tether or suture (or the like) to pass therethrough. In this way, the tether 150 may act as a tether or suture passer to translate another tether or suture to and/or through at least a portion (e.g., a suture-bone anchor) of the implant system.

As noted above and as shown in FIGS. 1-10, the implant system includes a dynamic bone anchor 100 through which the flexible suture or tether 150 extends. As also noted above, at least one other anchor (either an intraosseous anchor or a component configured to couple with a plate and facilitate routing of the tether 150 therethrough) is configured to be coupled to and implanted at least partially within a second bone, such as a bone of a syndesmotic joint (e.g., a distal fibula of a distal tibiofibular syndesmosis), with the tether 150 extending between and coupled to the dynamic bone anchor 100 and the at least one other bone anchor.

With reference to FIGS. 1-10, the dynamic bone anchor 100 comprises a cannulated main body portion 101. The cannulated main body portion 101 may include a proximal head portion 111, a distal end portion 103 and a body portion 102 extending between the body portion 102 and the proximal head portion 111. The body portion 101 comprises bone engagement projections 108 that extend about outer sides of the body portion 101. The head portion 11 may also comprise at least one such outer engagement projection 108. Likewise, the distal end portion 103 may or may not include the outer engagement projections 108.

The bone engagement projections 108 may be separate and distinct from each other, or may at least partially be continuous. In one embodiment, engagement projections 108 are concentrically extending teeth that angle or extend outwardly as they extend in a longitudinally direction extending from the distal end portion 103 to the head portion 111, as shown in FIGS. 1-10. In some other embodiments (not shown), the bone engagement projections 108 may comprise one or more threads, such as a helical thread.

The cannulation 110 of the main body portion 101 may extend through the proximal head portion 111, the distal end portion 103 and the body portion 102. The cannulation 110 extending through the head portion 111 may extend to a free end or top surface thereof (which may include a tool recess), thereby defining an opening to the cannulation 110 through which the tether 150 may extend, as shown in FIG. 10.

As shown in FIGS. 1-10, the main body portion 101 includes an outer groove 109 extending through the bone engagement projections 108. The outer groove 109 may be defined by an outer surface/face of the body portion 102, and potentially at least partially by an outer surface/face of the head portion 111 and/or the distal end portion 103.

As also shown in FIGS. 1-10, the distal end portion 103 includes a lateral through hole 114 that is longitudinally elongated and in communication with the inner cannulation 110. The lateral through hole 114 extends laterally entirely through a portion of the distal end portion 103 such that there are two openings to the cannulation 110 through the side wall of the distal end portion 103. The lateral through hole 114 forms a longitudinally elongated passageway laterally through the distal end portion 103.

The two openings of the lateral through hole 114 may be diametrically opposed, in one embodiment. Regardless of their arrangement, at least one of the ends/openings of the lateral through hole 114 is either substantially aligned with the outer groove 109 or spaced distally/below the bone engagement projections 108 such that the tether 150 is able to extend through the lateral through hole 114 and to/into the outer groove 109. Accordingly, the lateral through hole 114 may be in, aligned with or otherwise configured such that a clear pathway along the outer surface of the distal portion 103 (that is radially or beneath the outer ends of the bone engagement projections 108) extends longitudinally along the distal portion 103 to the outer groove 109.

Also shown in FIGS. 1-10, the body portion 102 includes a lateral aperture 112 that is in communication with the inner cannulation 110. The lateral aperture 112 extends laterally through a portion of the body portion 102 such that there is an opening that extends to the cannulation 110 through the side wall of the distal end portion 103. The lateral aperture 112 thereby forms a passageway laterally through a portion of the body portion 102 into the cannulation 110.

The lateral aperture 112 is substantially aligned with the outer groove 109 such that the tether 150 can extend along the outer groove 109 and into the cannulation 110 of the body portion 102 via the lateral aperture 112. In this way, the tether 150 can extend along the outer groove 109, into the cannulation 110 of the body portion 102, and out the opening at the end side of the head portion 111, as shown in FIG. 10. In some embodiments, the lateral aperture 112 is positioned in the outer groove 109.

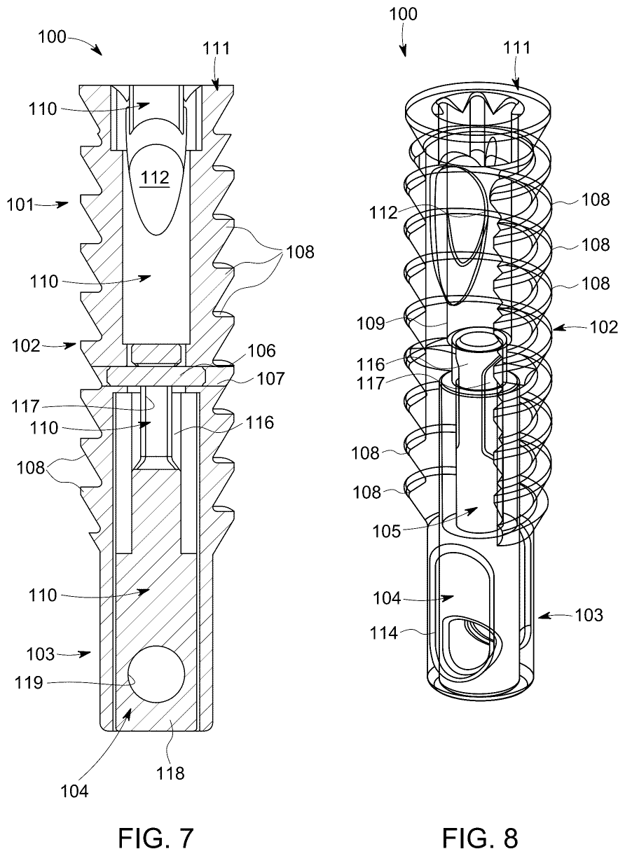

As shown in FIGS. 1-10, the dynamic bone anchor 100 may further comprise a piston member 104 and a resilient bumper 105 positioned and retained within the cannulation 110 of the main body 102. The bumper 105 may be positioned within the cannulation 110 of the body portion 102 between at least a portion of the piston member 104 and a retaining feature within the cannulation 110 of the body portion 102. In some embodiments, the retaining feature may comprise an interior wall of the body portion 102, as shown in FIG. 7 for example.

The piston member 104 is movably retained within the cannulation 110 of the main body portion 101. For example, a distal portion 116 of the piston member 104 may include a longitudinally elongated slot 117, as shown in FIGS. 1-10. The dynamic bone anchor 100 may include a member or portion 106 that extends into the longitudinally elongated slot 117 so that the piston member 104 is able to translate longitudinally within the cannulation 110 to the extent allowed by the elongated slot 117 and the member 106. In some embodiments, the member 106 may comprise a pin or like member that extends through a hole or aperture 107 in the wall of the body portion 102 of the main body portion 101, as shown in FIGS. 1-10.

In some embodiments, the distal portion 116 of the piston member 104 may be configured to extend longitudinally into/through/past the bumper 105 towards the head portion 111. For example, the distal portion 116 of the piston member 104 may be sized and shaped to extend longitudinally through/past a canulation of the bumper 105 such that the elongated slot 117 is longitudinally positioned between the bumper 105 and the head portion 111.

As shown in FIGS. 1-10, a distal base portion 118 of the piston member 104 may be configured to abut a distal longitudinal end or surface of the bumper 105. The bumper 105 may thereby be longitudinally trapped or contained within the cannulation 110 between the distal base portion 118 of the piston member 104 a feature (e.g., wall portion) of the main body portion 101.

The bumper 105 is longitudinally resilient/elastically deformable. The bumper 105 is at least axially/longitudinally compressible and elongatable within the cannulation 110. The bumper 105 may thereby be considered at least axially/longitudinally translatable within the cannulation 110 as at least some portions of the bumper 105 translate axially/longitudinally within the cannulation 110 as it axially/longitudinally compresses/shortens and elongates.

In some embodiments, the bumper 105 may be an elastic or resilient spring-like member. The bumper 105 may be comprised of one or more elastically deformable (e.g., resilient) members or material. For example, the bumper 105 may be one or more springs (e.g., disc or coil spring) or elastically compressible discs or tubes, or a combination thereof. For example, the bumper 105 may comprise an elastically compressible disc (e.g., elastomeric, polymer, polyurethane or polyethylene disc), tube (e.g., a polyurethane tube) or coil spring. In some embodiments, the bumper 105 comprises at least one urethane tube or like member, such as at least one polycarbonate urethane (PCU) tube or like member or at least one thermoplastic polyurethane (TPU) tube or like member.

The bumper 105 is configured such that it elastically deforms to tension the tether 150. As shown in FIGS. 1-10, the distal base portion 118 of the piston member 104 includes lateral through hole 119 that is in communication with the inner cannulation 110. The lateral through hole 119 extends laterally entirely through a portion of the distal base portion 118 such that there are two openings to the through hole 119. The lateral through hole 114 forms a passageway laterally through the distal base portion 118. Further, as shown in FIGS. 1-10, the lateral through hole 119 of the distal base portion 118 of the piston member 104 is at least partially aligned with the longitudinally elongated lateral through hole 114 of the distal portion 103. In some embodiments, the lateral through hole 119 and the lateral through hole 114 are fully aligned.

The lateral through hole 119 of the distal base portion 118 of the piston member 104 and the longitudinally elongated lateral through hole 114 of the distal portion 103 thereby form a passageway through the distal portion 103 of the main body 101. Further, as the lateral through hole 114 (and/or the lateral through hole 119) is longitudinally elongated, the piston member 104 is able to be forced longitudinally in the cannulation 110 toward the head portion 111 to compress the bumper 105 (e.g., to such a degree allowed/provided by the longitudinally elongated slot 117 and retaining member 106). In this way, a portion of the tether 150 may be laterally passed through the distal portion 103 via the lateral through hole 119 of the distal base portion 118 of the piston member 104 and the longitudinally elongated lateral through hole 114 of distal portion, and tensioned longitudinally toward the head portion 111 to resiliently compress the bumper 105 and apply a dynamic constraining and controlling tension force to the tether 150 to, for example, dynamically stabilize a joint and/or bones to which the anchor 100 and a secondary anchor 175 are coupled to (and coupled to the tether 150). The anchor 100 may thereby pull the anchor 100 and the secondary anchor 175 together, and/or allows a limited degree of relative movement between the anchor 100 and the secondary anchor 175.

The tether 150 may be fixed relative to the dynamic anchor 100 so that the resilient bumper 105 can be compressed and the preloaded tension thereby applied to the tether 150 via a portion of the tether being pinched or compressed between a bone 160 and the main body portion 101, such as the bone engagement projections 108, when the dynamic anchor 100 is implanted therein, as shown in FIGS. 9 and 10. For example, as shown in FIGS. 9 and 10, a portion of the tether 150 may extend longitudinally along the main body 101 over the bone engagement projections 108 between them and the bone 160 (and fixed therebetween), laterally through the lateral through holes 114, 117 to the side of the dynamic anchor 100 with the groove 109. Once at the side of the dynamic anchor 100 with the groove 109, the tether 150 may extend longitudinally along the outer groove 109 to the lateral aperture 112, into and longitudinally through the cannulation 100, and out of the top side opening of the cannulation 110 at the head portion 111.

As shown in FIGS. 18A-18D, the dynamic anchor 100 and tether 150 may be utilized with a variety of different additional bone-suture anchor devices 175, such as screws, caps, plates, soft anchors, etc., as described further below. Similarly, the dynamic anchor 100 may be implanted in any bone and/or bone location, and the tether 150 may extend to and coupled with one or more of the additional bone-suture anchor devices 175, which the dynamic anchor 100 providing the tension on the tether 150 (via the deformed bumper 105) to provide dynamic stabilization to the construct and bones/joint(s). For example, in some embodiments, the system may include a bone plate 175A may be secured to lateral fibula (e.g., via traditional screws and/or one or more anchor disclosed herein), one or more suture-bone anchor 100/175 secured to the fibula, and one or more suture-bone anchor 100/175 secured to the tibia, and the tether 150 extending through (in some fashion) and connected to the suture-bone anchors 100/175.

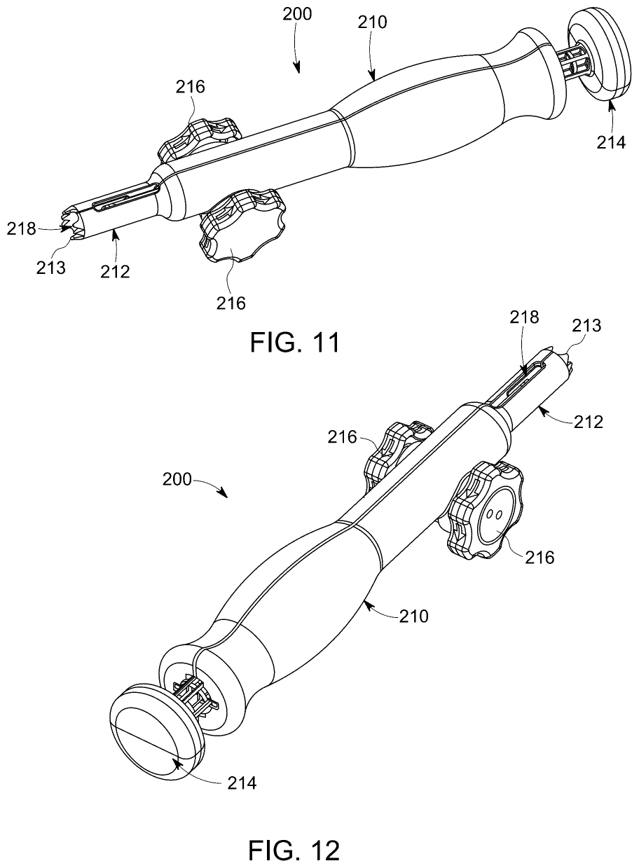

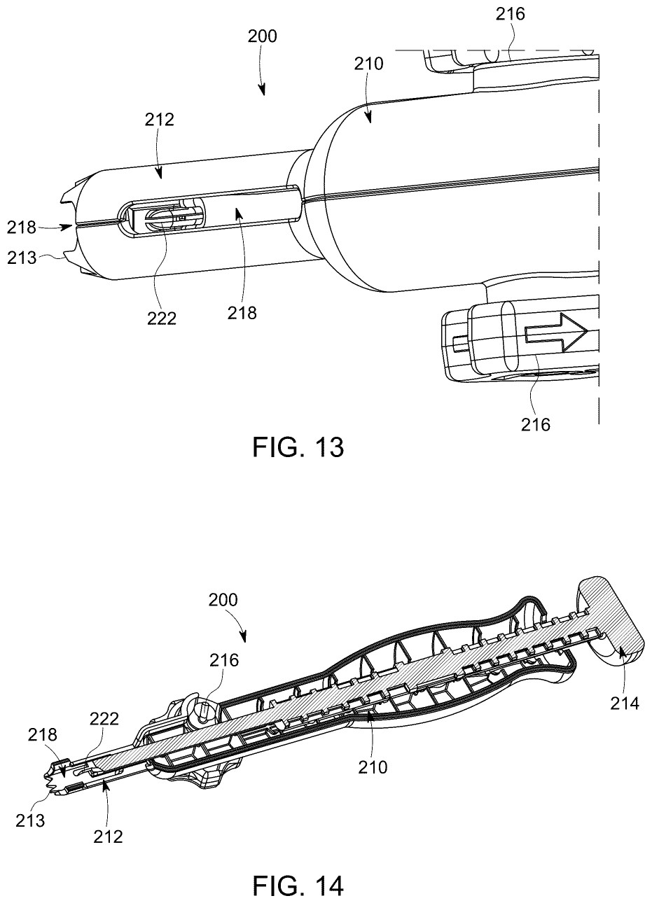

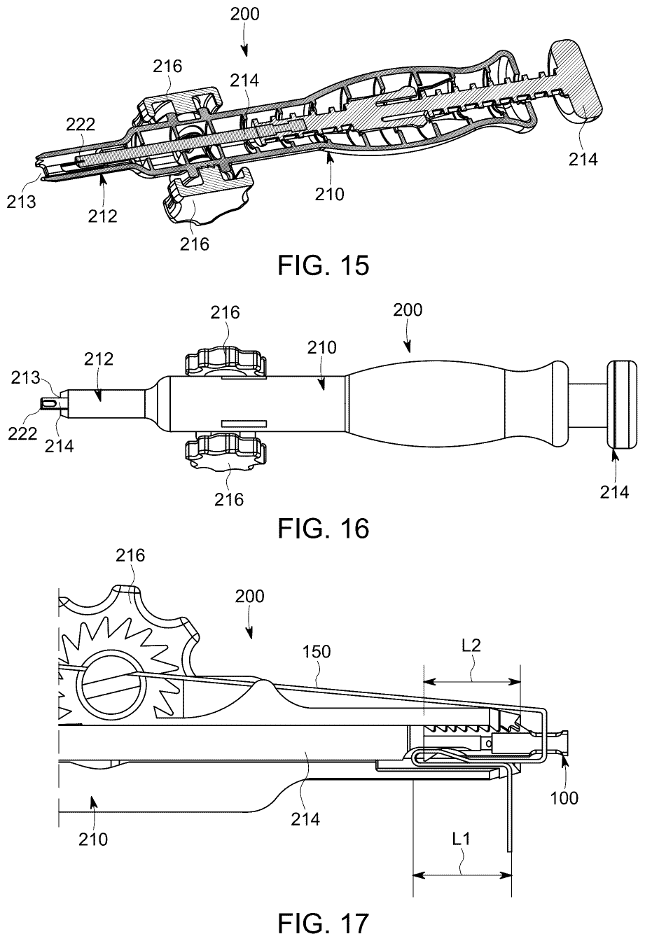

As shown in FIGS. 11-17, the stabilization system may include the dynamic anchor 100 and tether 150 coupled to an insertion or implant instrument 200 that is configured to implant and use the anchor 100 and tether 150. As shown in FIGS. 11-17, the dynamic anchor 210 may be removably housed within a cavity 218 of an insertion portion 212 of a main body 210 of the instrument 200, and the anchor 100 removably coupled on/over a coupler 222 at a distal end of an insertion handle 214. The insertion portion 212 may define an engagement end 213 that engaged a bone or a construct on a bone. In some embodiments, the engagement end 213 may include teeth or other structure to aid in friction between the insertion portion 212 and a bone.

The coupler 222 may be configured to releasably mate with the head portion 111 of the anchor 100, for example. The anchor 100 may be mounted on the coupler 22 and arranged within the insertion portion 212 such that at least a portion of the distal portion 103 extends longitudinally past the engagement end 213, as shown in FIGS. 16 and 17.

The insertion handle 214 may be manually longitudinally translatable or slidable within the main body 210 of the instrument 200 in a longitudinal direction extending toward the engagement end 213 to manually longitudinally translate the anchor 100 out from the insertion portion 212 and into a bone.

As also shown in FIGS. 11-17, the instrument 200 may include a tensioning handle or knob mechanism 216 rotatably mounted on the main body 210. The tether 150 may be coupled with or spooled on a portion of the tensioning mechanism 216 such that rotation of the tensioning mechanism 216 wraps the tether about a portion of the tensioning mechanism 216. In this way, tension may be applied to the tether 150 prior to implantation to preload the tension on the tether 150 via the resilient bumper 105, as discussed above. It is noted that length L1 of the portion of the tether 150 that extends from the dynamic bone anchor 100 to the engagement end 213 of the insertion portion 212 substantially equals a longitudinal length L2 of a portion of the dynamic bone anchor 100 that is housed in the cavity 218 of the insertion portion 214 from the engagement end 213 of the insertion portion 212. In this way, implantation of the anchor 100 via the handle 214 will not release or unload the preloaded tension of the tether 150.

After implantation, a portion of the tensioning mechanism 216 may be removed from the instrument 200 to expose the fixed portion of the tether 150 extending from between the anchor 100 and the bone. In this way, the additional length of tether 150 can be exposed and cut, if desired.

With reference to FIG. 19, the system may include at least one secondary suture-bone anchor configured as a soft deformable anchor 300. The soft deformable anchor 300 may be molded or formed via a solid material or a porous material/construct. As another example, the soft deformable anchor 300 may be woven from one or more threads, fibers or filaments. In some embodiments, the soft deformable anchor 300 may be formed via one or more machining, additive and/or extrusion formation process. The soft deformable anchor 300 may be of one-piece construction or formed of a plurality of members or portions (and/or materials). The soft deformable anchor 300 may be formed or made from any biocompatible material(s). For example, the soft deformable anchor 300 may be made or formed of polyester, polyethylene (e.g., ultra-high-molecular-weight polyethylene), polypropylene, nylon (e.g., polyamide), silk, polyglycolic acid (PGA), polydioxanone (PDO), polylactic acid (PLA), polylactic-co-glycolic acid (PLGA), stainless steel, or a combination thereof. In one example, the soft deformable anchor 300 is formed of polyester.

As explained above, a portion of the tether 150 may extend through the bone anchor 300. The bone anchor 300 may be a soft deformable bone anchor, such as an all-soft suture anchor (ASAs). For example, the bone anchor 300 may be a tube, sleeve or tape made of suture or tether material (e.g., woven) through which the tether 150 is woven. In some other embodiments, the tube, sleeve or tape may be solid and/or formed of a resilient material, such as an elastomer.

The soft deformable anchor 300 may be elongated along a longitudinal direction in a neutral state. In some embodiments, the soft deformable anchor 300 may define a longitudinal length within the range of 5 mm and 35 mm. The soft deformable anchor 300 may be a size proportion bigger than the tether 150. For example, the width or diameter (e.g., internal diameter) of the soft deformable anchor 300 may be larger than that of the tether 150.

In some embodiments, the soft deformable anchor 300 may be formed of an annular side wall that defines an inner cavity and first and second longitudinal ends. The soft deformable anchor 300 may thereby be hollow. The size of the inner cavity may be larger than the tether 150 such that the tether 150 is able to pass though the inner cavity. The inner cavity may extend through the soft deformable anchor 300 such that the inner cavity is open or accessible at one or both of the longitudinal ends, or the longitudinal ends may be closed or sealed. In some embodiments, the soft deformable anchor 300 may not define or include an inner cavity.

The soft deformable anchor 300 is flexible, collapsible, deformable, bendable, stretchable or otherwise re-arrangeable in overall shape. In a natural, neutral or non-deformed state, the soft deformable anchor 300 may extend substantially linearly or generally arcuately along the longitudinal direction. The soft deformable anchor 300 is configured such that tension on the portions on the suture extending from the soft deformable anchor 300 in a lateral direction that is angled from the longitudinal direction (e.g., substantially perpendicularly from the outer side surface of the anchor 300) deforms the soft deformable anchor 300 into a curved, bent-over, compressed or “bunched” shape, as shown in FIG. 28.

The tether 150 may extend through a side or portion of the soft deformable anchor 300 one or more times which, when tension in the lateral direction (at least generally), deforms the anchor 300 into a dense concave/convex, arcuate, folded, “C” or “U” and/or “V” shape as shown in FIG. 28. For example, in an installed or neutral state, the anchor 300 may be bent and/or pulled together (e.g., via the tether 150) such that it assumes a sharply arcuate (e.g., defined by a relatively small radius) U-shape or V-shape (not shown). The tether 150 may be tensioned laterally such that the tether 150 deforms, compresses or pulls together a side or portion of the soft deformable anchor 300 such that the deformable anchor 300 or a portion thereof bunches up, balls up or otherwise becomes more densely arranged in the sharply arcuate shape such that it defines a larger width, lateral/cross-sectional dimension, or other size. In this way, the soft deformable bone anchor 300 is configured such that when it is inserted into a properly sized/shaped hole, tunnel or cavity in a bone and the suture portions extending from the anchor 300 are tensioned/pulled at least generally laterally, the anchor 300 is cinched up and enlarged in at least one dimension such that the anchor 300 compress against the side wall of the hole/cavity in the bone, creating a dense “ball” that is squeezed within the hole/tunnel/cavity and, thereby, anchored therein. As explained further below, in some exemplary methods, for dynamic stabilization of a distal tibiofibular syndesmosis, a hole/tunnel/cavity can be formed in the distal tibia (which may be formed via drilling the distal tibia). The anchor 300 may thereby be a tibial anchor. It is noted that the configuration of the anchor 300 allows it to be placed in a bone hole/tunnel/cavity with a substantially smaller diameter (e.g., 1-3 mm), thereby preserving bone and allowing for more bone preservation, for example.

With reference to FIG. 20, the system may include at least one secondary suture-bone anchor configured as a static suture-bone bone-engagement anchor 400. The static bone-engagement anchor 400 may be configured to couple directly within a bone, and not with a bone plate or other bone-intermediary construct.

In some embodiments, the static bone-engagement anchor 400 may comprise a threaded shaft portion 413 and a head portion 411 that is configured to be directly seated within/on bone, rather than a bone plate for example. The head portion 411 includes a cavity or through hole 410 through which the tether 150 can be passed. The head portion 411 may thereby act as a cross-member that statically retains and affixes the tether 150 to a portion of a bone in which the shaft portion 413 implanted. In one exemplary embodiment, the static bone-engagement anchor 400 may be a static bone-engagement anchor disclosed in in International PCT Application No. PCT/US2022/013302.

With reference to FIG. 21, the system may include at least one secondary suture-bone anchor configured as a static suture-bone swivel anchor 500. The swivel anchor 500 may be configured to couple directly within a bone (and not with a bone plate or other bone-intermediary construct), or may be configured to couple with a bone plate or other bone-intermediary construct that is coupled to a bone (and potentially the bone as well).

In some embodiments, the static bone-engagement anchor 500 may comprise a threaded shaft portion 513 and a head portion 511 that includes a socket with a swivel member 515 that is retained therein and able rotate therein. The swivel member 515 may be able to pivot about a central portion of the head 511 so as to provide angular adjustability of the path of the suture/tether 150. The head portion 511 may be configured to be directed seated within/on bone, or configured to engage a bone plate for example. The swivel member 515 includes a cavity or through hole 510 through which the tether 150 can be passed. The swivel member 515 may thereby act as a cross-member that statically retains and affixes the tether 150 to a portion of a bone in which the shaft portion 513 implanted.

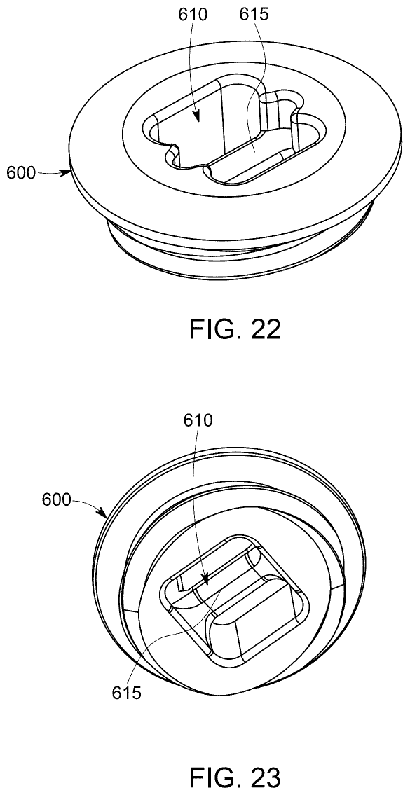

With reference to FIGS. 22 and 23, the system may include at least one secondary suture-bone anchor configured as a static suture-plate interfacing cap anchor 600. The static cap anchor 600 may be configured to couple (and potentially lock) with a bone plate or other bone-intermediary construct that is coupled to a bone, and not extend directed into the bone.

In some embodiments, the suture-plate interfacing cap anchor 600 may comprise a threaded shaft portion that is configured to threadably mate within an aperture of a bone plate, and not extend into the bone. The threaded shaft portion may or may not be configured to threadably lock with the bone plate.

As shown in FIGS. 22 and 23, the suture-plate interfacing cap anchor 600 may also comprise a head portion that includes a cavity or through hole 610 with a cross-member 615 or other structure that extends (at least partially) across or through the cavity or through hole 610. The tether 150 can thereby pass through the cavity 610 and under or around the cross-member 615 or other structure to couple the tether 150 to the cap anchor 600. The cross-member 615 may thereby statically retain and affix the tether 150 to a bone plate which is attached to a bone. In one exemplary embodiment, the suture-plate interfacing cap anchor 600 may be a suture-plate interfacing anchor disclosed in in International PCT Application No. PCT/US2021/017888.

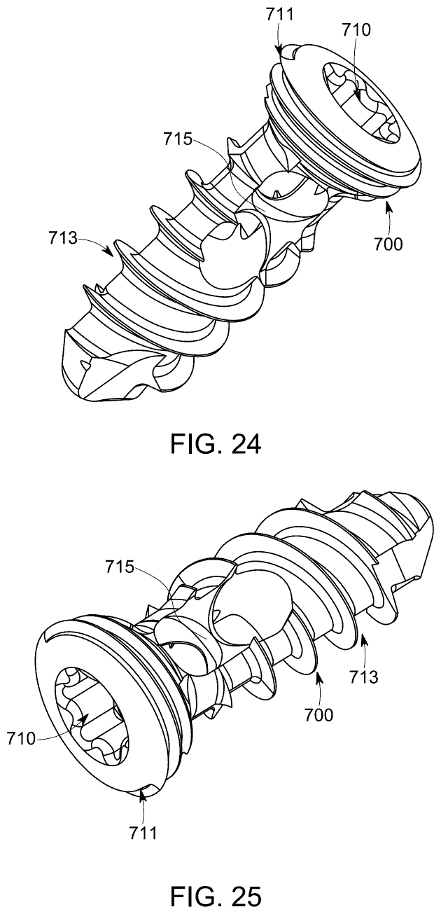

With reference to FIGS. 24 and 25, the system may include at least one secondary suture-bone anchor configured as a static suture-plate interfacing bone anchor 700. The static suture-plate interfacing bone anchor 700 may be configured to couple (and potentially lock) with a bone plate or other bone-intermediary construct that is coupled to a bone, and also extend directed into the bone.

In some embodiments, the suture-plate interfacing bone anchor 700 may comprise a threaded head portion 711 that is configured to threadably mate within an aperture of a bone plate that is coupled to a bone, and a threaded shaft portion 713 that is configured to be implanted into the bone. The threaded head portion 711 may or may not be configured to threadably lock with the bone plate. In some other embodiments, head portion 711 may not be threaded.

As shown in FIGS. 24 and 25, the suture-plate interfacing bone anchor 700 includes a cannulation 710 that extends into the head portion 711 into/through which the tether 150 can be passed. The bone anchor 700 may further include a cross-member or other structure 715 in, or in communication with, the cannulation 710. In some embodiments, the cross-member or other structure 715 may extend across the cannulation 710. The tether 150 can passed through the cannulation 710 and under or around the cross-member 715 or other structure to couple the tether 150 to the bone anchor 700. The cross-member 715 may thereby statically retain and affix the tether 150 to a bone plate and bone to which the bone anchor 700 is attached/coupled.

As another example, the system may include at least one secondary suture-bone anchor configured as a bone plate, such as a bone plate the same as or similar as, but not limited to, the bone plate 175A illustrated in FIGS. 18A-18B. In some embodiments, the system may be implemented in conjunction with a bone plate and one or more other implants having static or dynamic suture retaining peripeties, and configured to be implanted to and/or at least partially in at least one bone (e.g., the tibia and/or fibula).

As may be recognized by those of ordinary skill in the art based on the teachings herein, numerous changes and modifications may be made to the above-described and other embodiments of the present disclosure without departing from the scope of the disclosure. In addition, the implants and systems may include more or fewer components or features than the embodiments as described and illustrated herein. Accordingly, this detailed description of the currently-preferred embodiments is to be taken as illustrative, as opposed to limiting the disclosure.

Similarly, positions or directions may be used herein with reference to anatomical structures or surfaces. Further, the implants, systems, devices, instrumentation and methods, and the aspects, components, features and the like thereof, may be disclosed herein are described with respect to one side of the body for brevity purposes. However, as the human body is relatively symmetrical or mirrored about a line of symmetry (midline), it is hereby expressly contemplated that the implants, systems, devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems, instrumentation and methods, and the aspects, components, features and the like thereof, described herein with respect to a right syndesmosis joint (right ankle/leg) may be mirrored or otherwise reconfigured, if necessary or desirable, so that they likewise function with a left syndesmosis joint (left ankle/leg). Further, the implants, systems, devices, instrumentation and methods, and the aspects, components, features and the like thereof, disclosed herein are described with respect to a distal tibiofibular syndesmosis,, but it should be understood that the implants, systems, devices, instrumentation and methods may be used with other bones of the body having similar structures.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An implant system for dynamic stabilization of bones and/or tissue, comprising:
a dynamic bone anchor; and
at least one tether extending through and between the dynamic bone anchor and at least one secondary tether anchor,
wherein the dynamic bone anchor comprises:
a cannulated main body comprising:
a proximal head portion, a distal end portion and a body portion extending between the proximal head portion and the distal end portion;
a piston member positioned within the cannulation of the main body; and
a resilient bumper positioned within cannulation of the body portion between at least a portion of the bumper and a retaining feature within the cannulation of the body portion,
wherein the body portion comprises bone engagement projections extending about outer sides thereof,
wherein the body portion comprises an outer groove extending through the bone engagement projections,
wherein the piston member comprises a first lateral through hole, and the distal end portion comprises a second lateral through hole that is in communication with the first lateral through hole, and
wherein the body portion comprises a lateral aperture that is in communication with the cannulation thereof and is substantially aligned with the outer groove.

2. The system of claim 1, wherein the lateral aperture is positioned within the outer groove.

3. The system according to claim 1, wherein the first lateral through hole is substantially aligned with the outer groove.

4. The system according to claim 1, wherein the proximal head portion comprises at least one bone engaging projection extending about outer sides thereof.

5. The system according to claim 1, wherein the distal end portion is void of a bone engaging projection.

6. The system according to claim 1, wherein the piston member is movably retained within the cannulation of the main body.

7. The system according to claim 6, wherein a distal portion of the piston member comprises an elongated slot, and wherein a pin member extends from a wall portion of the main body into the elongated slot to limit the travel of the piston member through the cannulation.

8. The system according to claim 7, wherein the distal portion of the piston member extends into cannulation of the bumper.

9. The system according to claim 1, wherein the retaining feature comprises an interior wall of the body portion.

10. The system according to claim 9, wherein the resilient bumper is positioned within cannulation of the body portion between a distal portion of the bumper and the interior wall.

10. The system according to claim 1, wherein the cannulation of the main body extends to top side of the head portion such that the cannulation forms a top side opening at the top side of the head portion that is in communication with the cannulation and the lateral aperture.

11. The system according to claim 10, wherein a portion of the tether extends through the top side opening, through the cannulation of the main body to the lateral aperture, through the lateral aperture, and along the outer groove to the second lateral through hole.

12. The system according to claim 1, wherein a portion of the tether extends along the main body over the bone engagement projections, laterally through a first side of the second lateral through hole, laterally through the first lateral through hole, and laterally through a second side of the second lateral through hole.

13. The system according to claim 1, wherein the system comprises a plurality of the dynamic bone anchors.

14. The system according to claim 1, wherein the at least one secondary tether anchor comprises a plurality of secondary tether anchors.

15. The system according to claim 14, wherein the at least one secondary tether anchor comprises at least one soft bone anchor that is configured to deform into an enlarged shape upon tensioning of the tether extending thereto.

16. The system according to claim 15, wherein the at least one secondary tether anchor comprises at least one bone plate.

17. The system according to claim 16, wherein the at least one secondary tether anchor further comprises at least one anchor cap configured to couple with an aperture of the at least one bone plate, the at least one anchor cap comprising an open cavity with at least one tether retention cross-member within the open cavity.

18. The system according to claim 16, wherein the at least one secondary tether anchor comprises at least one static bone anchor configured to couple with an aperture of the at least one bone plate, the at least one static bone anchor comprising an open cavity with at least one tether retention cross-member within the open cavity.

19. The system according to claim 1, wherein the at least one secondary tether anchor comprises at least one secondary static bone anchor configured to couple directly within a bone and not with a bone plate, wherein the at least one secondary static bone anchor comprises an open cavity with at least one tether retention cross-member within the open cavity.

20. The system according to claim 1, wherein the tether comprises a plurality of tethers, and wherein at least one the plurality of tethers is configured as a tether passer comprising at least one loop or through hole.

21. The system according to claim 1, further comprising an instrument coupled with a dynamic bone anchor of the at least one dynamic bone anchor configured to insert the dynamic bone anchor into a bone.

22. The system according to claim 21, wherein the tether is coupled to a tensioning handle of the instrument that is removably and rotatably coupled to a housing body, and wherein the instrument is configured such that rotation of the tensioning handle wraps the tether about a portion of the tensioning handle and, facilitates tensioning the tether.

23. The system according to claim 21, wherein the tether extends through the first and lateral through holes, and wherein the tether extends from the cannulation of the head portion between the dynamic bone anchor and an insertion portion of the instrument that houses the dynamic bone anchor and out a distal end of the insertion portion.

24. The system according to claim 23, wherein the length of the portion of the tether that extends between the dynamic bone anchor and the insertion portion to the distal end of the insertion portion substantially equals a longitudinal length of a portion of the dynamic bone anchor that is housed in the insertion portion from the distal end of the insertion portion.

25. A method of dynamically stabilizing a distal tibiofibular syndesmosis joint, comprising:
obtaining the implant system of claim 1;
implanting the dynamic bone anchor into at least one of a fibula or a tibia of the joint; and
implanting the at least one secondary tether anchor into the other of the fibula or the tibia,
wherein implanting the dynamic bone anchor comprises tensioning the tether between the dynamic bone anchor and the at least one secondary tether anchor to dynamically stabilize the joint.
